(19) United States
(12) Patent Application Publication
(10) Pub. No.: US 2026/0241153 A1
(43) Pub. Date: Aug. 20, 2026

(54) SKIN TREATMENT DEVICE USING SKIN ELECTROPORATION FOR DRUG DELIVERY

(71) Applicant: CHUNGWOO MEDICAL CO., LTD., Seoul (KR)

(72) Inventors: IL-KWON LEE, Anyang-si (KR); KEE-SEOK LEE, Seoul (KR); SEONG-YI PARK, Seoul (KR)

(21) Appl. No.: 19/193,391

(22) Filed: Apr. 29, 2025

(30) Foreign Application Priority Data
Feb. 19, 2025 (KR) 10-2025-0021820
Mar. 12, 2025 (KR) 10-2025-0031932

(51) Int. Cl. A61M 37/00; A61M 39/24; A61N 1/04; A61N 1/32

(52) U.S. Cl. CPC A61M 37/00; A61M 39/24; A61N 1/327; A61M 2037/0007; A61M 2205/106; A61M 2205/123; A61N 1/0412

(57) ABSTRACT

The present disclosure relates to a skin treatment device using skin electroporation for drug delivery, including: a case member having a drug outlet; a drug container separably received in the case member; a pump coupled to a front side discharge hole of the drug container to take a drug out of the drug container; a pump-operating part located inside the case member to operate the pump so that the pump takes the drug out of the drug container; an electrode part for applying electrical stimulation to a skin tissue to allow the drug to be delivered to the skin tissue if the drug is discharged to the skin tissue through the drug outlet; and a signal generator for generating a signal for the electrical stimulation of the electrode part.

### CROSS REFERENCE TO RELATED APPLICATION OF THE DISCLOSURE

The present application claims the benefit of Korean Patent Application No.10-2025-0031932 filed in the Korean Intellectual Property Office on March, 12, 2025, the entire contents of which are incorporated herein by reference.

### BACKGROUND OF THE DISCLOSURE

### Field of the Disclosure

The present disclosure relates to a skin treatment device, more specifically to a skin treatment device using skin electroporation for drug delivery that is capable of being configured to easily inject a drug directly to a skin tissue according to a movement of a pump-operating part and to allow a drug container to be replaceable.

### Background of the Related Art

Generally, drug delivery to a skin tissue is favored for its many advantages, as an attractive clinical method. The skin is the largest organ of the human body, easily accessible and observable, and is an immune-competent organ. However, the non-permeability of skin and the barrier function of skin are main obstacles in efficient transdermal drug delivery.

The skin of the human body has a surface area of about 2 m and an average thickness of about 2 mm, which of course has an individual difference. The skin has two main tissue types: the epidermis and the dermis.

The epidermis as the continually keratinizing stratified epithelium has the stratum corneum that is the outermost layer thereof and serves as a primary barrier for skin.

The stratum corneum contains 15 to 30 layers of corneocytes not visible and biochemically active, and all layers of the epidermis, the stratum granulosum, the stratum spinosum, and the stratum basale contain keratinocytes, immune Langerhans cells, and dermal dendritic cells in different steps of differentiation.

As physical and chemical methods for transdermal drug delivery, there are iontophoresis, lipid delivery, and gene gun, which are well known, and further, another physical method for increasing the permeability of a drug into skin is electroporation.

The electroporation is the application of short electrical pulses creating pores in a lipid bilayer membrane of a mammalian cell. The electroporation enables macromolecules such as a drug hard to permeate the skin in methods different therefrom to permeate the skin. The electroporation increases the absorption of drug into the skin as well as the degree of delivery of drug into a target tissue. To perform the electroporation creating the cores through which the drug passes, threshold energy should be required, and movements generated by electrophoretic effect depend on both of electrical fields and pulse lengths.

After the drug has been applied to the skin through a separate device, however, the electroporation is performed to apply the electrical pulses to a surgery area, thereby causing inconvenient processes and a lot of time for surgery. Therefore, there is a need to combine the individual devices with each other under a structure not complicated.

As the skin treatment device using drug injection performs a direct treatment onto a surgery area, further, it advantageously has high effectiveness, easy and fast surgery, and no side effect such as infection due to small invasion. In spite of such advantages, it is hard to allow the drug to permeate the skin due to the existence of stratum corneum cells, which is the biggest difficulty of the skin treatment device using drug injection, and therefore, various methods have been proposed to overcome such disadvantages.

A drug injection syringe, which is generally used in conventional practices, is configured to allow a drug stored in a drug container to be pushed by a plunger to discharge an amount of drug required. In this case, an amount of drug injected is determined according to the moving distance of the plunger, and therefore, such a drug injection method is generally applied to syringes.

Another method for discharging a given amount of liquid is a pumping method in which a head with an outlet is pressed to suck a drug due to a pressure difference generated when a take-out space becomes small and then to discharge the drug through the operations of outlet and inlet valves, and the pumping method is widely applied for various liquids such as shampoo, cosmetics, and the like. In the case of the pumping method, an amount of liquid taken out from a liquid container is determined according to the depth and speed of the head pressed and the remaining amount of liquid, so that it is difficult to take out an accurate amount of liquid from the liquid container. Further, the liquid remains in a take-out path and on the bottom of the liquid container, so that it is hard to take out a precise amount of liquid from the liquid container and the pumping method is thus difficult to be applied to a high-priced drug or a drug easy to be denatured.

The electroporation is a physical treatment technique that effectively delivers a drug to the skin or mucous membrane. It uses electrical pulses to create pores in cell membranes, allowing the drug to be absorbed into the skin or mucous membrane. In the process, as the drug directly permeates a specific area, the electroporation has fewer side effects than oral medication and syringe injection and better effectiveness than them.

Main indications for drug using the electroporation are muscle or joint pain, inflammation, skin diseases, scars, whitening for beauty, and a specific target area to which a local treatment is performed to deliver a drug thereto, thereby preventing the drug from being applied to the entire body.

If a skin treatment device for drug injection is used by individual users who are not skilled, there is no problem in the injection of the entire drug at a time, but if the drug is injected again after a given amount of drug has been injected, the drug may remain in an injection path, thereby causing the loss of drug and the denaturization of the remaining drug. Such problems are difficult and inconvenient to be treated even in specialized hospitals, and therefore, it is harder for individual users to treat the problems.

Further, conventional drug containers are not equipped with application devices as required according to use purposes of drugs, and most of them are made to pack and distribute the drugs. Therefore, there is a need to develop a drug application device that combines a drug container with electroporation.

Furthermore, an electroporation device is bulky in size and complicated in structure, and therefore, the electroporation device is recognized as professionally used in hospitals or specialized surgery institutions, not as used by individuals. As materials or surgeries for skin treatment or skincare are widely known to the general public, recently, individual desires to skin surgery increase. To do this, devices for application of materials have been developed, but an electroporation device for an individual use or a portable skin treatment device has not been suggested yet.

Besides, it is hard that a conventional drug supply device supplies an accurate amount of drug under the use of pressure or the movement of a plunger, and further, the drug remains in a supply path to cause contamination due to its denaturization, thereby definitely needing a solution to such problems.

### PRIOR ART DOCUMENT

### Patent Documents

1) Korean Patent No. 10-0220615 (Sep. 15, 1999)
2) Korean Patent No. 10-1113001 (Feb. 24, 2012)

### SUMMARY OF THE DISCLOSURE

Accordingly, the present disclosure has been made in view of the above-mentioned problems occurring in the related art, and it is an object of the present disclosure to provide a skin treatment device using skin electroporation for drug delivery that is capable of being configured to allow a drug to be delivered directly to a skin tissue according to a movement of a pump-operating part and to have a drug container replaceable to prevent the drug from remaining in a drug take-out pipe and a discharge pipe, so that as simply surgery is completed within a short time anytime and anywhere, during daily life, working, or moving, the drug delivery and the drug absorption through electrical stimulation are performed quickly and easily.

To accomplish the above-mentioned objects, according to the present disclosure, there is provided a skin treatment device using skin electroporation for drug delivery including: a case member having a drug outlet; a drug container separably received in the case member; a pump coupled to a front side discharge hole of the drug container to take a drug out of the drug container; an electrode part for applying electrical stimulation to a skin tissue to allow the drug to be delivered to the skin tissue if the drug is discharged to the skin tissue through the drug outlet; and a signal generator for generating a signal for the electrical stimulation of the electrode part.

According to the present disclosure, desirably, the skin treatment device may further include a pump-operating part having one end connected to a moving body of the pump and the other end connected to the drug outlet inside the case member, and the pump-operating part may operate the pump through a linear movement by a given distance so that the pump takes the drug stored in the drug container by a predetermined amount to allow the drug to be directly delivered to the skin tissue through the drug outlet.

According to the present disclosure, desirably, the case member may include: a body case as a hollow structure in which the drug container is separably received, the body case having a coupling hole and first and second guide ribs for guiding the operation of the pump-operating part; cover cases coupled to both ends of the body case in such a way as to surround both sides of the drug container exposed to the outside; and a nozzle case coupled to the other end of the body case and having the drug outlet thereon.

According to the present disclosure, desirably, the body case is constituted of first and second semicircular body cases coupled to each other.

According to the present disclosure, desirably, the cover cases are coupled integrally to one end and the other end of the first body case and one end and the other end of the second body case.

According to the present disclosure, desirably, the second body case may include a coupling boss and a partition plate having a through hole thereon.

According to the present disclosure, desirably, the nozzle case may be coupled to a nozzle block having a discharge pipe connected to the drug outlet and a first tube connection pipe adapted to pass through the through hole of the partition plate.

According to the present disclosure, desirably, one side cover case may have an air inlet adapted to introduce air into the body case, if the pump operates by means of a linear movement of the pump-operating part and thus takes the drug out of the drug container.

According to the present disclosure, desirably, the drug container may have a piston moving therein in such a way as to open a front side discharge hole thereof according to the operation of the pump-operating part to allow the drug stored therein to be discharged therefrom, so that as the piston moves therein, the rear volume of the drug container is filled with the air introduced through the air inlet.

According to the present disclosure, desirably, one side cover case may be rotatably coupled to the body case, and the body case may have a drug discharge amount indicator with three graduations, so that as one side cover case has a coupling position reference protrusion protruding therefrom in such a way as to be brought into contact with the pump-operating part, the range of the linear movement of the pump-operating part is determined according to a degree of rotation of the coupling position reference protrusion to allow the amount of drug taken out of the drug container to be adjusted.

According to the present disclosure, desirably, the pump may include: a moving body connected to the drug container through a drug take-out pipe in such a way as to move interlockingly with the linear movement of the pump-operating part; a first spring compressed according to the movement of the moving body; a discharge piston pressurizedly moving according to the movement of the moving body; and a first check valve located in the drug take-out pipe in such a way as to open the drug take-out pipe if the discharge piston pressurizedly moves, whereby an amount of drug corresponding to a moving distance of the discharge piston is discharged from the drug take-out pipe.

According to the present disclosure, desirably, the discharge piston may return to an original position thereof by means of a restoring force of the first spring compressed.

According to the present disclosure, desirably, the pump may further include a second check valve located in the drug take-out pipe in such a way as to be opened, by means of the increase of pressure generated when the discharge piston 33 returns to the original position thereof, to transmit the increasing pressure to the drug take-out pipe, whereby the drug remaining in the drug take-out pipe is discharged.

According to the present disclosure, desirably, the first check valve and the second check valve may have the same configuration as each other, and each check valve may include a spring providing an elastic force with respect to a partition wall located inside the drug take-out pipe and a ball supported against the spring in such a way as to guide the flow of drug or air with a given pressure from the opposite direction to the spring in one direction, while closing an opening of the partition wall.

According to the present disclosure, desirably, the pump-operating part may include: a first operating block coupled to the coupling hole of the body case in such a way as to allow a portion thereof to be exposed to the outside of the body case so that if pressed, the first operating block linearly moves in a first direction along the first guide rib; a second operating block having one end coupled to the pump and the other end connected to the drug outlet in such a way as to linearly move in a second direction perpendicular to the first direction if the first operating block linearly moves in the first direction, to allow the pump to operate to take the drug out of the drug container; a guide block fixedly fastened to the coupling boss of the second body case by means of a screw to guide the linear movement of the second operating block in the second direction; and a tube having one end connected to the second operating block and the other end connected to the first tube connection pipe of the nozzle block to guide the drug taken out of the drug container to the drug outlet.

According to the present disclosure, desirably, the first operating block may include: a press button partially exposed to the outside of the coupling hole of the body case; and operating rods coupled to the press button, one of the operating rods having a third guide rib protruding therefrom in such a way as to correspond to the first guide rib so that if the press button is pressed, the operating rods linearly move in the first direction along the first guide rib, and the operating rods may have first slant portions formed on the ends thereof in such a way as to allow the second operating block to linearly move in the second direction.

According to the present disclosure, desirably, the press button may have a slip prevention groove thereon.

According to the present disclosure, desirably, the second operating block may include: a pump joint body linearly moving in the second direction and having a guide hole on one end thereof in such a way as to pass the guide block therethrough and a guide groove corresponding to the second guide rib; a second slant portion formed on top of the pump joint body in such a way as to be brought into close contact with the first slant portions to linearly move the pump joint body in the second direction if the operating rods linearly move in the first direction; a pump connector located on one end of the pump joint body in such a way as to be connected to the pump to operate the pump if the pump joint body linearly moves in the second direction, so that the drug is taken out of the drug container; and a second tube connection pipe located on the other end of the pump joint body in such a way as to be connected to the tube to guide the drug taken through the pump connector to the tube.

According to the present disclosure, desirably, the tube may have a bent portion adapted to allow a fastened state thereof to be kept consistently by means of a repulsion force, thereby preventing the drug from leaking to the outside.

According to the present disclosure, desirably, the electrode part may consist of an upper electrode and a counter electrode as independent electrodes located on the nozzle case and apply electric current to the user's skin located between the upper electrode and the counter electrode.

According to the present disclosure, desirably, the upper electrode and the counter electrode may be cylindrical structures made of conductive materials and having different diameters from each other, each electrode having a spring at the center thereof to provide a conductive circuit through which the contact with the skin tissue is kept.

According to the present disclosure, desirably, the signal generator may include: a circuit board for generating an electrical signal if a power button is pressed; a battery for supplying driving power to the circuit board; and a charging socket for charging the battery, wherein the circuit board supplies a monopolar or bipolar electroporation signal to the upper electrode and the counter electrode connected thereto through a connection line according to the pressing of the power button.

According to the present disclosure, desirably, the circuit board may be connected to a hand-held ground electrode, and the monopolar or bipolar electroporation signal may be transmitted between the upper electrode and the counter electrode.

According to the present disclosure, desirably, the power button may be coupled to the case member by means of hooks and have an illuminator thereon.

### BRIEF DESCRIPTION OF THE DRAWINGS

The above and other objects, features and advantages of the present disclosure will be apparent from the following detailed description of the embodiments of the disclosure in conjunction with the accompanying drawings, in which:

FIG. 1 is a perspective view showing a skin treatment device using skin electroporation for drug delivery according to an embodiment of the present disclosure;

FIG. 2 is an exploded perspective view showing the skin treatment device according to the embodiment of the present disclosure;

FIG. 3 is an exploded perspective view showing a state wherein a case member and a drug container are coupled to each other in the skin treatment device according to the embodiment of the present disclosure;

FIG. 4 is a partially cut away perspective view showing a state wherein a tube is not connected to the case member in the case where the drug container is received in the case member according to the embodiment of the present disclosure;

FIG. 5 is a partially cut away perspective view showing a state wherein the tube is connected to the case member in the case where the drug container is received in the case member according to the embodiment of the present disclosure;

FIGS. 6A and 6B are perspective views showing a first operating block of the skin treatment device according to the embodiment of the present disclosure;

FIGS. 7A and 7B are perspective views showing a second operating block of the skin treatment device according to the embodiment of the present disclosure;

FIG. 8 is a perspective view showing a guide block of the skin treatment device according to the embodiment of the present disclosure;

FIG. 9 is an enlarged perspective view showing a state before a pump-operating part connected to a nozzle block through the tube operates according to the embodiment of the present disclosure;

FIG. 10 is an enlarged perspective view showing a state after the pump-operating part connected to the nozzle block through the tube operates according to the embodiment of the present disclosure;

FIGS. 11A and 11B are enlarged perspective views showing a state wherein the first operating block is coupled to a second body case according to the embodiment of the present disclosure;

FIG. 12 is a perspective view showing the tube having a bent portion according to the embodiment of the present disclosure;

FIG. 13 is a schematic sectional view showing a state wherein the drug container is coupled to the case member according to the embodiment of the present disclosure;

FIG. 14 is a schematic enlarged sectional view showing a coupling structure between a drug take-out pipe and check valves according to the embodiment of the present disclosure;

FIG. 15 is a schematic sectional view showing the check valve according to the embodiment of the present disclosure;

FIG. 16 is an enlarged perspective view showing a state wherein a signal generator is located in the case member according to the embodiment of the present disclosure;

FIG. 17 is a perspective view showing a skin treatment device using skin electroporation for drug delivery according to another embodiment of the present disclosure, wherein the skin treatment device has a rotatable cover case;

FIG. 18 is an exploded perspective view showing the skin treatment device according to another embodiment of the present disclosure;

FIG. 19 is an exploded perspective view showing a state wherein a drug container is coupled to a case member according to another embodiment of the present disclosure;

FIG. 20 is a partially cut away perspective view showing a state wherein a tube is not connected to the case member in the case where the drug container is received in the case member according to another embodiment of the present disclosure;

FIG. 21 is a partially cut away perspective view showing a state wherein the tube is connected to the case member in the case where the drug container is received in the case member according to another embodiment of the present disclosure;

FIG. 22 is an exploded view showing a signal generator according to another embodiment of the present disclosure; and

FIGS. 23 and 24 are partially cut away perspective views showing a state wherein the signal generator is received in the case member according to another embodiment of the present disclosure.

### DETAILED DESCRIPTION OF THE PREFERRED EMBODIMENT

Hereinafter, an embodiment of the present disclosure will be explained with reference to the attached drawings.

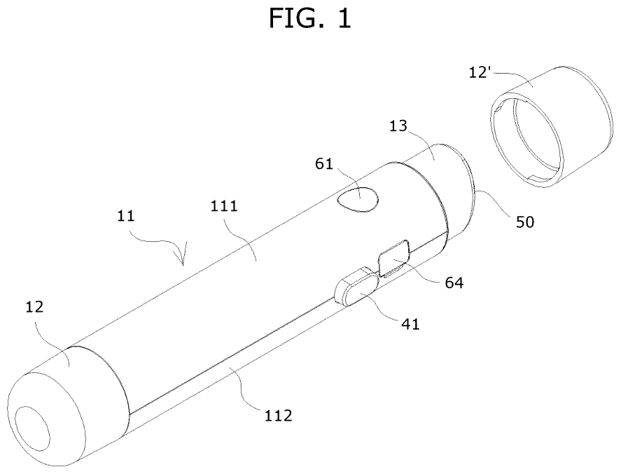

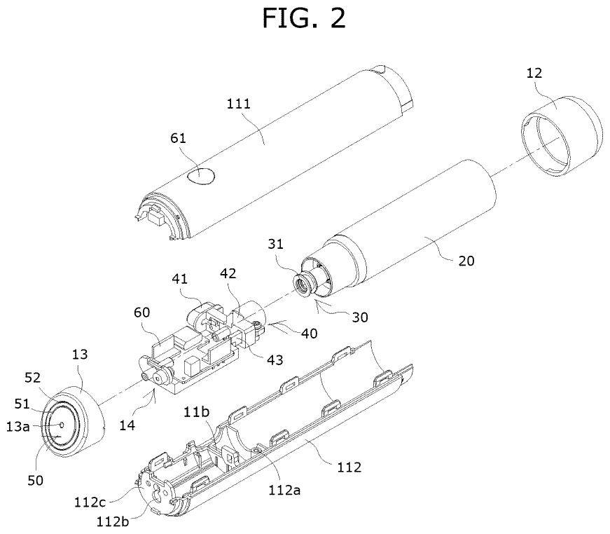

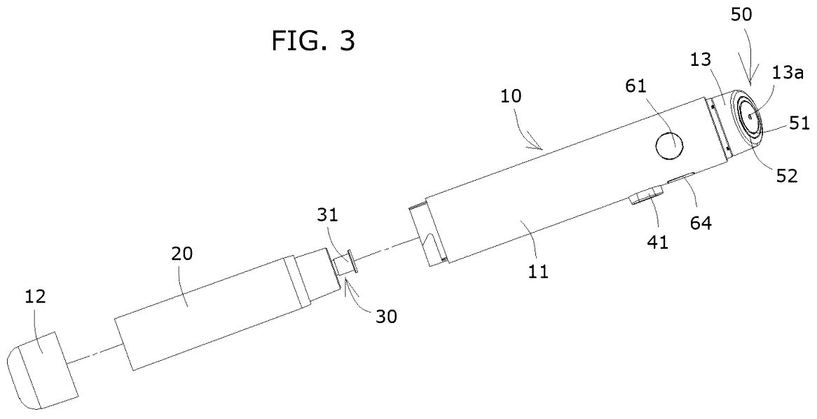

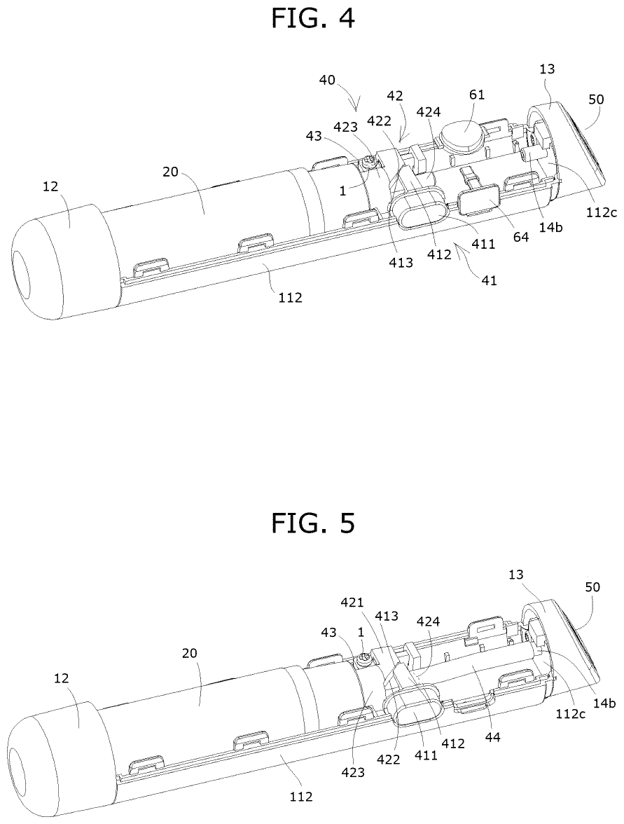

FIG. 1 is a perspective view showing a skin treatment device using skin electroporation for drug delivery according to an embodiment of the present disclosure, FIG. 2 is an exploded perspective view showing the skin treatment device according to the embodiment of the present disclosure, FIG. 3 is an exploded perspective view showing a state wherein a case member and a drug container are coupled to each other in the skin treatment device according to the embodiment of the present disclosure, FIG. 4 is a partially cut away perspective view showing a state wherein a tube is not connected to the case member in the case where the drug container is received in the case member according to the embodiment of the present disclosure, and FIG. 5 is a partially cut away perspective view showing a state wherein the tube is connected to the case member in the case where the drug container is received in the case member according to the embodiment of the present disclosure.

Referring to FIGS. 1 to 5, a skin treatment device using skin electroporation for drug delivery according to an embodiment of the present disclosure largely includes a case member 10, a drug container 20, a pump 30, a pump-operating part 40, an electrode part 50, and a signal generator 60.

The case member 10 is portable and consists of a body case 11, cover cases 12 and 12′, and a nozzle case 13.

The body case 11 is a hollow structure in which the drug container 20 is received separably therefrom and consists of a coupling hole 11a and first and second guide ribs 11b and 11c for guiding the operation of the pump-operating part 40.

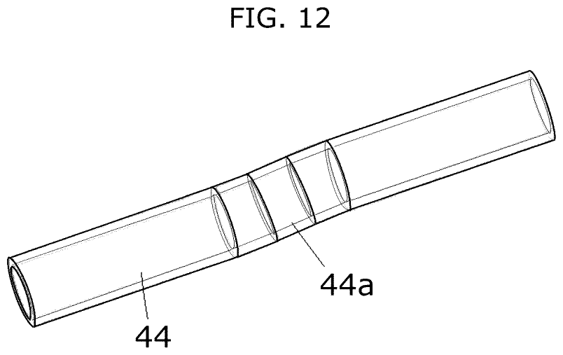

In this case, the body case 11 is constituted of a pair of first and second semicircular body cases 111 and 112 facing each other, and to allow the first and second body cases 111 and 112 to be coupled to each other, as shown in FIGS. 11A and 11B, the second body case 112 is provided with a coupling boss 112a and a partition plate 112c with a through hole 112b.

The cover cases 12 and 12′ are coupled to both ends of the body case 11 in such a way as to surround both sides of the drug container 20 exposed to the outside when the drug container 20 is received in the body case 11, and the cover case 12 coupled to one side of the body case 11 has an air inlet 15 adapted to introduce air into the body case 11 if the pump 30 operates by means of a linear movement of the pump-operating part 40 and thus takes a drug out of the drug container 20.

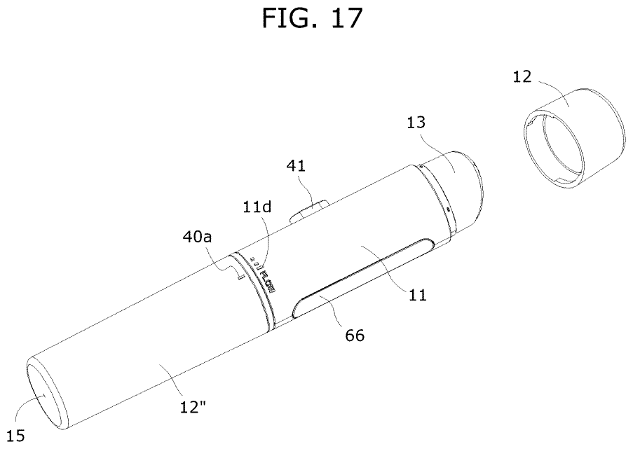

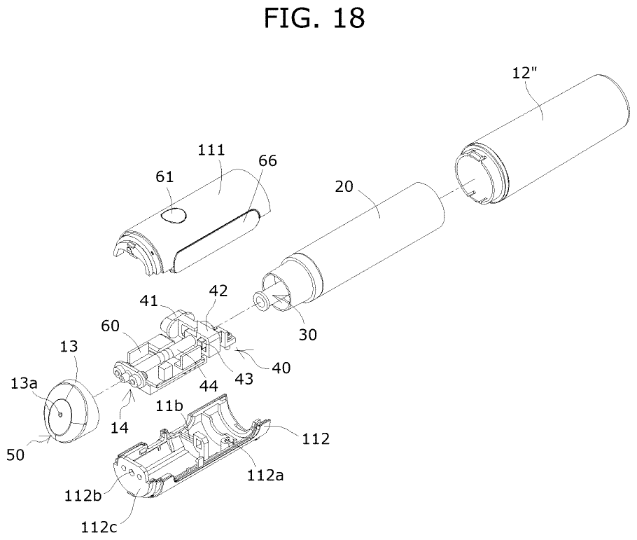

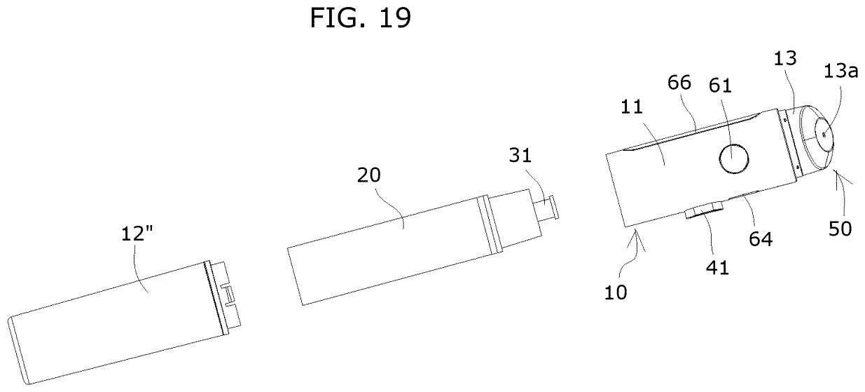

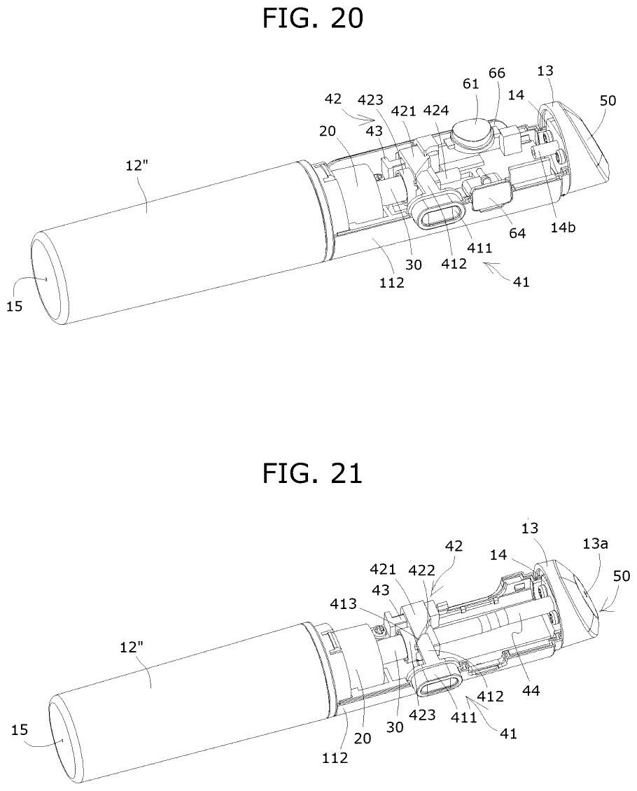

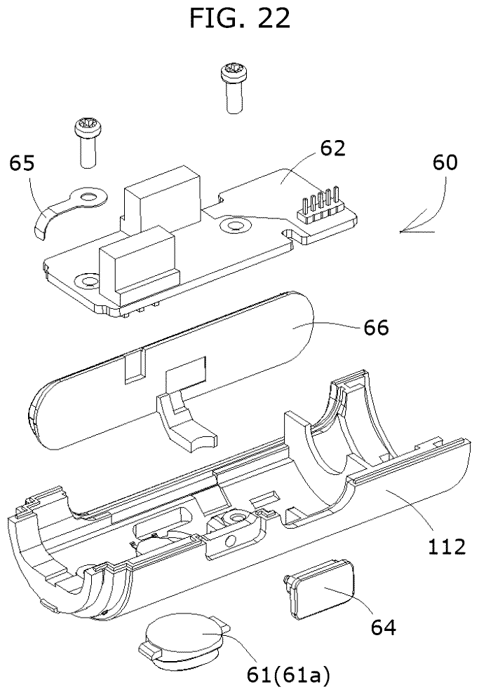

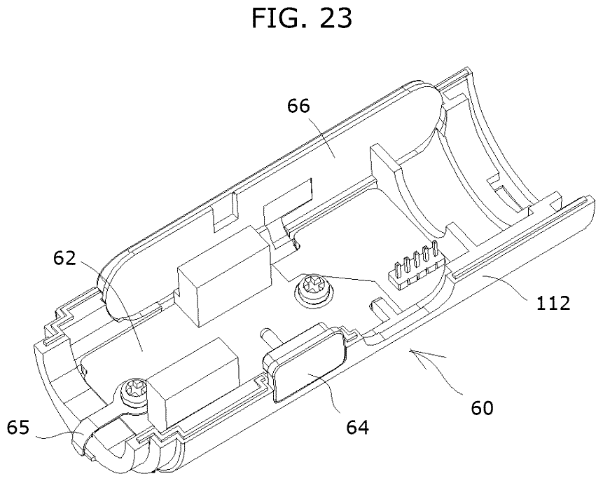

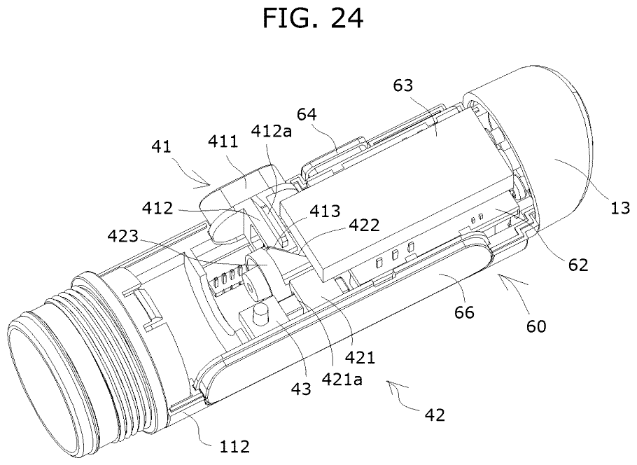

Further, the cover case 12′ is coupled integrally to one end of the first semicircular body case 111 and one end of the second semicircular body case 112, and otherwise, a cover case 12″ as shown in FIGS. 17 to 24 is rotatably coupled to the body case 11. In the case where the cover case 12″ is rotatably coupled to the body case 11, a drug discharge amount indicator 11d having three graduations is formed on the body case 11, and a coupling position reference protrusion 40a protrudes from the cover case 12″ in such a way as to be brought into contact with the pump-operating part 40, so that the range of the linear movement of the pump-operating part 40 is determined according to a degree of rotation of the coupling position reference protrusion 40a to allow the amount of drug taken out of the drug container 20 to be adjusted.

That is, as a linearly moving distance of the pump-operating part 40 is limited by the rotation of the coupling position reference protrusion 40a, the amount of drug taken out of the drug container 20 is adjustable.

The nozzle case 13 is coupled to the other end of the body case 11 and has a drug outlet 13a.

In this case, the nozzle case 13 is coupled to a nozzle block 14, and the nozzle block 14 has a discharge pipe 14a connected to the drug outlet 13a and a first tube connection pipe 14b adapted to pass through the through hole 113b formed on the partition plate 112c.

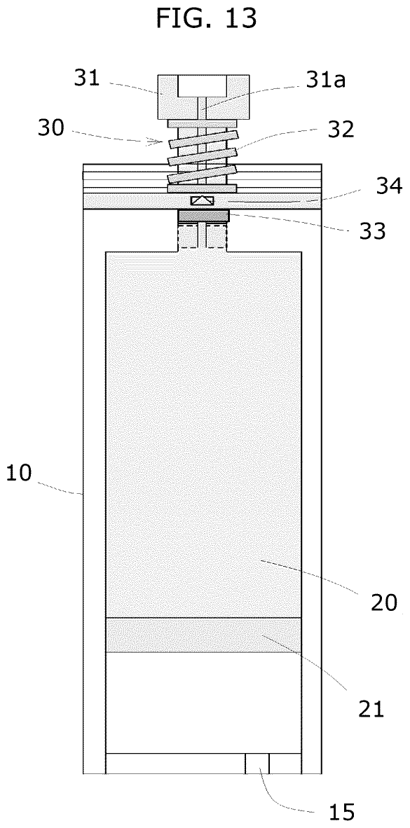

The drug container 20 is separably received in the case member 10, and as shown in FIG. 13, the drug container 20 has a piston 21 moving therein in such a way as to open a front side discharge hole (with no reference numeral) thereof according to the operation of the pump-operating part 40 to allow the drug stored therein to be discharged therefrom. As the piston 21 moves, further, the rear volume of the drug container 20 is filled with the air introduced by the air inlet 15.

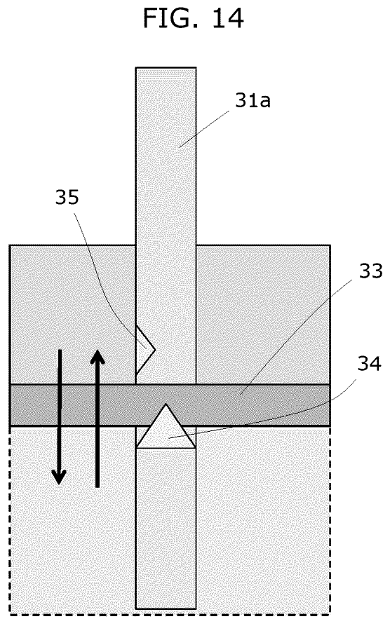

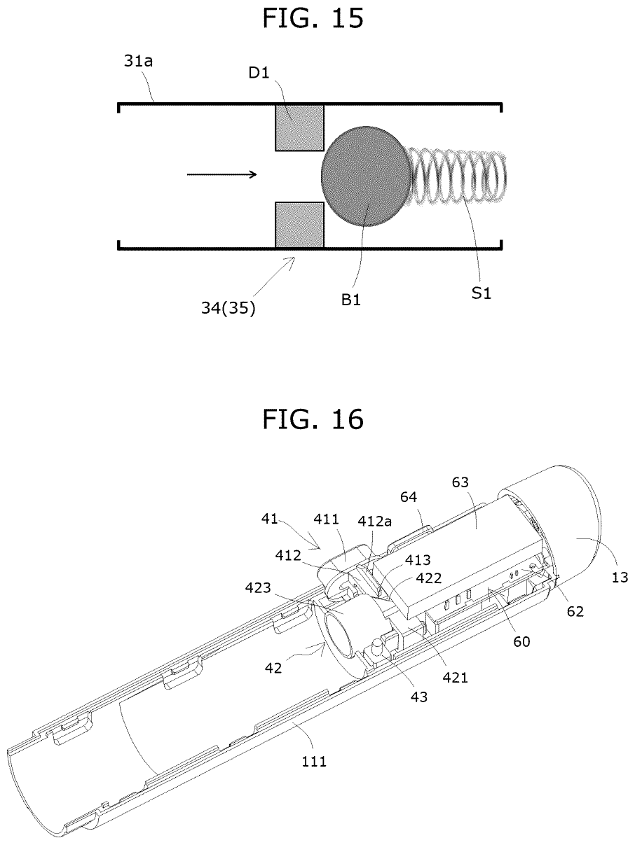

The pump 30 is coupled to the front side discharge hole o the drug container 20 and operates to take the drug out of the drug container 20. As shown in FIGS. 13 to 15, the pump 30 includes a moving body 31, a first spring 32, a discharge piston 33, a first check valve 34, and a second check valve 35.

The moving body 31 is connected to the drug container 20 through a drug take-out pipe 31a and moves interlockingly with the linear movement of the pump-operating part 40.

The first spring 32 is compressed according to the movement of the moving body 31 and provides a restoring force for allowing the moving body 31 to return to its original position if a given amount of drug is taken out of the drug container 20.

The discharge piston 33 pressurizedly moves according to the movement of the moving body 31, and the first check valve 34 is located in the drug take-out pipe 31a in such a way as to open the drug take-out pipe 31a if the discharge piston 33 pressurizedly moves.

Accordingly, the drug is discharged from the drug take-out pipe 31a by the moving distance of the discharge piston 33.

The second check valve 35 is located in the drug take-out pipe 31a in such a way as to be open, by means of the increase of pressure generated when the discharge piston 33 returns to its original position, to thus transmit the increasing pressure to the drug take-out pipe 31a, thereby allowing the drug remaining in the drug take-out pipe 31a to be discharged.

In this case, the first and second check valves 34 and 35 have the same configuration as each other, and as shown in FIG. 15, each check valve includes a spring S1 providing an elastic force with respect to a partition wall D1 located inside the drug take-out pipe 31a and a ball B1 supported against the spring S1 in such a way as to guide the flow of drug or air with a given pressure from the opposite direction to the spring S1 only in one direction, while closing an opening of the partition wall D1.

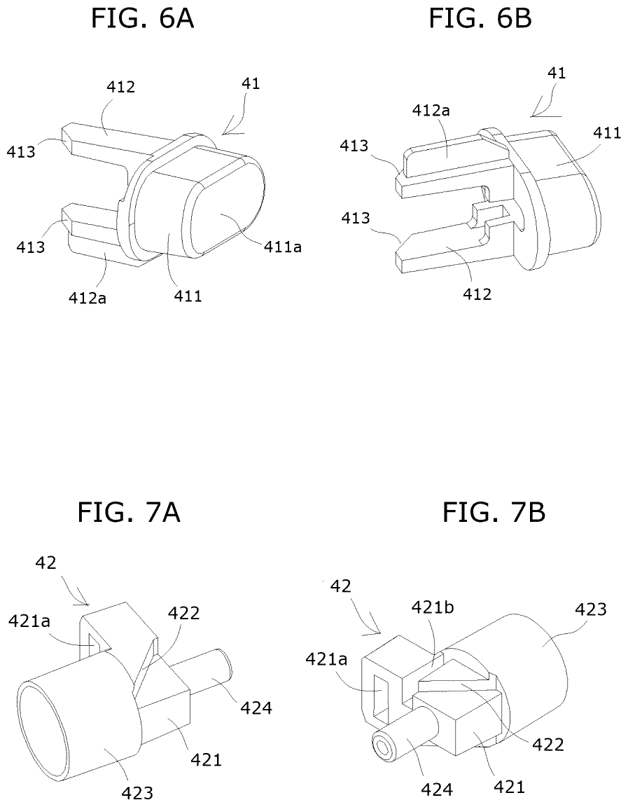

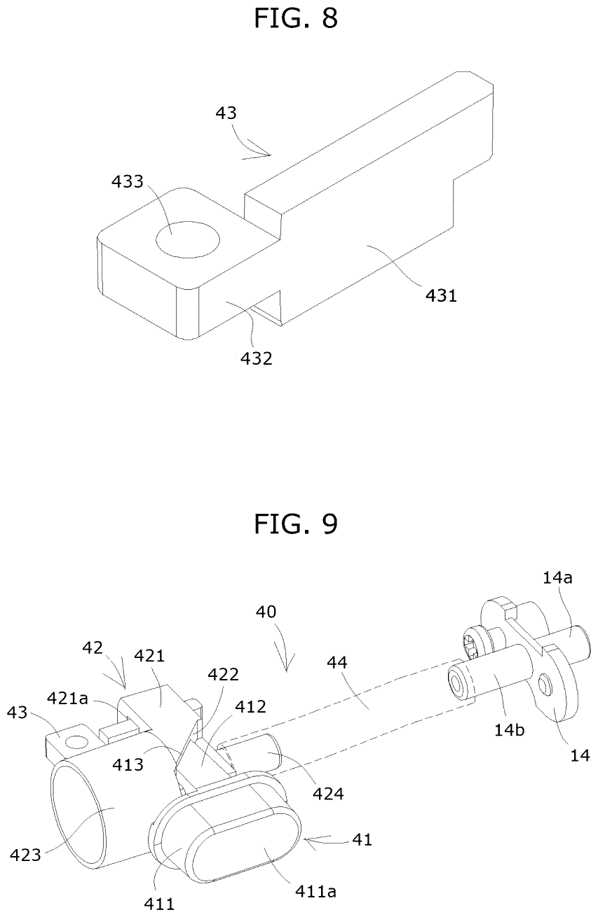

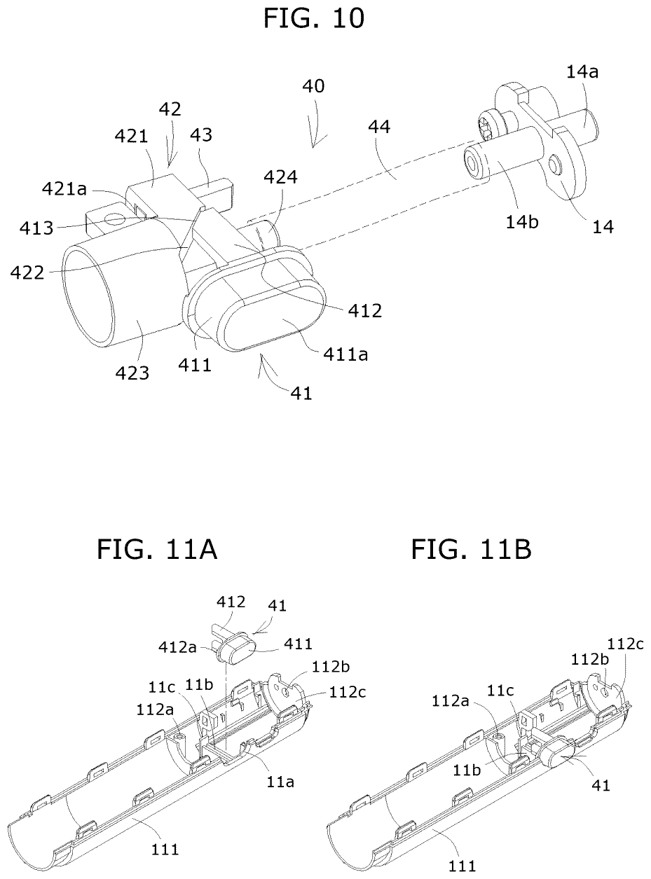

The pump-operating part 40 has one end connected to the moving body 31 of the pump 30 and the other end connected to a tube 44 as will be discussed later, inside the case member 10, and the pump-operating part 40 operates the pump 30 through the linear movement by a given distance so that the pump 30 takes the drug stored in the drug container 20 by a predetermined amount to allow the drug to be directly delivered to a skin tissue through the drug outlet 13a of the nozzle case 13. As shown in FIGS. 6A to 11, the pump-operating part 40 includes a first operating block 41, a second operating block 42, a guide block 43, and the tube 44.

The first operating block 41 is coupled to the coupling hole 11a of the body case 11 in such a way as to allow a portion thereof to be exposed to the outside of the body case 11, and if the first operating block 41 is pressed, it linearly moves in a first direction (that is, in a transverse direction) along the first guide rib 11b. The first operating block 41 includes a press button 411, operating rods 412, and first slant portions 413.

The press button 411 is partially exposed to the outside of the coupling hole 11a of the body case 11 in such a way as to be pressed by a user and has a slip prevention groove 411a formed thereon to prevent slip from occurring when pressed.

The operating rods 412 protrude from the press button 411, and one of the operating rods 412 has a third guide rib 412a protruding therefrom in such a way as to correspond to the first guide rib 11b, so that if the press button 411 is pressed, the operating rods 412 linearly move in the first direction along the first guide rib 11b.

The first slant portions 413 are formed on the ends of the operating rods 412 in such a way as to allow the second operating block 42 to linearly move in a second direction (in a longitudinal direction) perpendicular to the first direction.

The second operating block 42 has one end coupled to the pump 30 and the other end connected to the drug outlet 13a and linearly moves in the second direction perpendicular to the first direction, if the first operating block 41 linearly moves in the first direction, to allow the pump 30 to operate to take the drug out of the drug container 20. The second operating block 42 includes a pump joint body 421, a second slant portion 422, a pump connector 423, and a second tube connection pipe 424.

The pump joint body 421, which linearly moves in the second direction to operate the pump 30, has a guide hole 421a on one end thereof in such a way as to pass the guide block 43 therethrough and a guide groove 421b corresponding to the second guide rib 11c.

The second slant portion 422 is formed on top of the pump joint body 421 in such a way as to be brought into close contact with the first slant portions 413 and serves to linearly move the pump joint body 421 in the second direction if the operating rods 412 linearly move in the first direction.

That is, the linearly moving distance of the pump joint body 421 is determined by means of the contact area of the second slant portion 422 with the first slant portions 413, and further, the pumping distance of the pump 30 is also determined. Under such determinations, a degree of openness of the discharge hole of the drug container 20 is determined, thereby allowing an amount of drug taken out of the drug container 20 to be determined.

The pump connector 423 is located on one end of the pump joint body 421 in such a way as to be connected to the pump 30, and if the pump joint body 421 linearly moves in the second direction, the pump connector 423 operates the pump 30 to allow the drug to be taken out of the drug container 20.

The second tube connection pipe 424 is located on the other end of the pump joint body 421 in such a way as to be connected to the tube 44 and serves to guide the drug taken through the pump connector 423 to the tube 44.

The guide block 43 is fixedly fastened to the coupling boss 112a of the second body case 112 by means of a screw 1 and serves to guide the pump joint body 421 to linearly move in the second direction.

The tube 44 has one end connected to the second tube connection pipe 424 of the second operating block 42 and the other end connected to the first tube connection pipe 14b of the nozzle block 14 and serves to guide the drug taken out of the drug container 20 to the drug outlet 13a. Further, as shown in FIG. 12, the tube 44 has a bent portion 44a adapted to allow a fastened state thereof to be kept consistently by means of a repulsion force, thereby preventing the drug from leaking to the outside.

The electrode part 50 serves to apply electric stimulation to the skin tissue, if the drug is discharged to the skin tissue through the drug outlet 13a, to allow the discharged drug to be applied to the skin tissue by means of electroporation, and to do this, the electrode part 50 consists of an upper electrode 51 and a counter electrode 52 as independent electrodes located on the nozzle case 13. Therefore, the electrode part 50 applies electric current to the user's skin located between the upper electrode 51 and the counter electrode 52.

In this case, the upper electrode 51 and the counter electrode 52 are cylindrical structures made of conductive materials and having different diameters from each other, and further, they have springs (not shown) at the centers thereof to provide conductive circuits through which the contacts with the skin tissue are kept.

The signal generator 60 serves to generate an electrical signal for electrical stimulation for the electrode part 50 and includes a circuit board 62 for generating the electrical signal if a power button 61 is pressed, a battery 63 for supplying driving power to the circuit board 62, and a charging socket 64 for charging the battery 63. The circuit board 62 serves to supply a monopolar or bipolar electroporation signal to the upper electrode 51 and the counter electrode 52 connected thereto through a connection line 65 according to the pressing of the power button 61.

In detail, as a hand-held ground electrode 66 is connected to the circuit board 62, the monopolar or bipolar electroporation signal is transmitted between the upper electrode 51 and the counter electrode 52, and the power button 61 is coupled to the case member 10 by means of hooks and has an illuminator 61a.

Under such a configuration, as shown in FIGS. 1 to 24, according to the embodiment of the present disclosure allows the electrode part 50 located on one surface of the nozzle case 13 to be brought into close contact with the user's skin tissue in a state where the body case 11 is grasped by him or her.

Next, if the press button 411 of the first operating block 41 of the pump-operating part 40 located on the side of the body case 11 is pressed, the operating rods 412 of the first operating block 41 linearly move in the first direction along the first guide rib 11b and the third guide rib 412a.

In this case, as the first slant portions 413 formed on the ends of the operating rods 412 are brought into close contact with the second slant portion 422 of the second operating rod 42, the pump joint body 421 of the second operating block 42 linearly moves in the second direction perpendicular to the first direction along the guide block 43 and the second guide rib 11c if the operating rods 412 linearly move in the first direction, so that the moving body 31 of the pump 30 linearly moves toward the drug container 20 under the linear movement of the pump joint body 421.

In this case, the first spring 32 is compressed upon the linear movement of the moving body 31, and further, the discharge piston 33 linearly moves interlockingly with the moving body 31, so that the first check valve 34 located in the drug take-out pipe 31a is opened. As a result, the discharge hole of the drug container 20 that is blocked by means of the first check valve 34 is opened to allow the drug in the drug container 20 to be taken out through the drug take-out pipe 31a, and the drug taken out of the drug container 20 is discharged by fixed amount to the user's skin tissue through the tube 44, the discharge pipe 14a of the nozzle block 14, and the drug outlet 13a.

In this case, if the power button 61 is pressed by the user to supply the driving power of the battery 63 to the circuit board 62, the circuit board 62 supplies the monopolar or bipolar electroporation signal to the electrode part 50 brought into the skin tissue through the connection line 65, so that the electrode part 50 applies the drug discharged through the drug outlet 13a to the skin tissue by means of the electroporation.

If the drug is discharged to the skin tissue through the drug outlet 13a, the discharge piston 33 returns to its original position by means of the restoring force of the first spring 32 compressed, so that a pressure of the closed space in the body case 11 increases to allow the second check valve 35 located in the drug take-out pipe 31a to be opened. As the second check valve 35 is opened, the pressure is transmitted to the drug take-out pipe 31a, so that the drug remaining in the drug take-out pipe 31a is completely discharged, thereby preventing the remaining drug from being denatured and contaminated.

As described above, the skin treatment device using skin electroporation for drug delivery according to the present disclosure is configured to allow the drug to be delivered directly to the skin tissue according to the movement of the pump-operating part, to prevent the drug from remaining in the drug take-out pipe and the discharge pipe, and to have the drug container replaceable, thereby expecting the following effectiveness.

Firstly, an accurate amount of drug can be discharged, irrespective of the remaining amount of drug.

Secondly, the drug can be used up until the amount of drug remaining in the drug container disappears, without any loss, thereby achieving economical advantages.

Thirdly, no drug remains in the drug take-out pipe and the discharge pipe, and therefore, the drug can be prevented from being denatured and contaminated, thereby achieving conveniences and safety.

Lastly, the drug is delivered, and simultaneously, the drug penetrates into the skin tissue by means of the electroporation, so that as two individual steps are performed simultaneously, the surgery is completed simply within a short time, and as the skin treatment device is portable, the user can use it anytime and anywhere, thereby allowing the surgery to be performed during daily life, working, or moving.

The effectiveness of the invention is not limited as mentioned above, and it should be understood to those skilled in the art that the effectiveness of the invention may include another effectiveness as not mentioned above from the detailed description of the present invention.

The foregoing description of the embodiments of the present disclosure has been presented in the specification and drawings. In the description of the present invention, special terms are used not to limit the present invention and the scope of the present invention as defined in claims, but just to explain the present invention.

While the present disclosure has been made with reference to the particular illustrative embodiments, it is not to be restricted by the embodiments but only by the appended claims. It is to be appreciated that those skilled in the art can change or modify the embodiments without departing from the scope and spirit of the present disclosure.

What is claimed is:

1. A skin treatment device using skin electroporation for drug delivery, comprising:
a case member having a drug outlet;
a drug container separably received in the case member;
a pump coupled to a front side discharge hole of the drug container to take a drug out of the drug container;
a pump-operating part located inside the case member to operate the pump so that the pump takes the drug out of the drug container;
an electrode part for applying electrical stimulation to a skin tissue to allow the drug to be delivered to the skin tissue if the drug is discharged to the skin tissue through the drug outlet; and
a signal generator for generating a signal for the electrical stimulation of the electrode part.

2. The skin treatment device according to claim 1, wherein the case member comprises:
a body case as a hollow structure in which the drug container is separably received, the body case having a coupling hole and first and second guide ribs for guiding the operation of the pump-operating part;
cover cases coupled to both ends of the body case in such a way as to surround both sides of the drug container exposed to the outside; and
a nozzle case coupled to the other end of the body case and having the drug outlet thereon.

3. The skin treatment device according to claim 2, wherein on the side surface of the body case is located a press button so that while the drug is being discharged, the press button is prevented from pressing a user's skin.

4. The skin treatment device according to claim 1, wherein the drug container has a piston moving therein in such a way as to open the front side discharge hole according to the operation of the pump-operating part to allow the drug stored therein to be discharged therefrom, so that as the piston moves therein, the rear volume of the drug container is filled with the air introduced by an air inlet formed on the cover case coupled to one side of the body case.

5. The skin treatment device according to claim 1, wherein the pump comprises:
a moving body connected to the drug container through a drug take-out pipe in such a way as to move interlockingly with the linear movement of the pump-operating part;
a first spring compressed according to the movement of the moving body;
a discharge piston pressurizedly moving according to the movement of the moving body; and
a first check valve located in the drug take-out pipe in such a way as to open the drug take-out pipe if the discharge piston pressurizedly moves,
whereby an amount of drug corresponding to the moving distance of the discharge piston is discharged from the drug take-out pipe.

6. The skin treatment device according to claim 5, wherein the discharge piston returns to an original position thereof by means of a restoring force of the first spring compressed.

7. The skin treatment device according to claim 5, wherein the pump further comprises a second check valve located in the drug take-out pipe in such a way as to be opened, by means of the increase of pressure generated when the discharge piston returns to the original position thereof, to transmit the increasing pressure to the drug take-out pipe, whereby the drug remaining in the drug take-out pipe is discharged.

8. The skin treatment device according to claim 7, wherein the first check valve and the second check valve have the same configuration as each other, and each check valve comprises a spring providing an elastic force with respect to a partition wall located inside the drug take-out pipe and a ball supported against the spring in such a way as to guide the flow of drug or air with a given pressure from the opposite direction to the spring in one direction, while closing an opening of the partition wall.

9. The skin treatment device according to claim 3, wherein the press button has a slip prevention groove thereon.

10. The skin treatment device according to claim 2, wherein the electrode part consists of an upper electrode and a counter electrode as independent electrodes located on the nozzle case and applies electric current to the user's skin located between the upper electrode and the counter electrode.

11. The skin treatment device according to claim 10, wherein the upper electrode and the counter electrode are cylindrical structures made of conductive materials and having different diameters from each other, each electrode having a spring at the center thereof to provide a conductive circuit through which the contact with the skin tissue is kept.
